(19) United States
(12) Patent Application Publication
(10) Pub. No.: US 2022/0020477 A1
(43) Pub. Date: Jan. 20, 2022

(54) Real Time Scheduling Tool and Method of Use

(71) Applicant: YUTAN, LLC

(72) Inventor: Daniel Martinez, Sugar Land, TX (US)

(21) Appl. No.: 16/931,562

(22) Filed: Jul. 17, 2020

(51) Int. Cl. G16H 40/20; G06Q 10/10; H04W 4/14

(52) U.S. Cl. CPC G16H 40/20; H04W 4/14; G06Q 10/1095

(57) ABSTRACT

A system and method for real time updating of scheduling events in preferably professional, medical and dental applications.

### CROSS-REFERENCE TO RELATED APPLICATIONS

Not Applicable.

### STATEMENT REGARDING FEDERALLY SPONSORED RESEARCH

Not applicable.

### BACKGROUND

The present invention relates to systems and methods for real time updating of appointment schedules in any professional setting, including medical, dental and other applications. In present society, it would be beneficial for patients to have real time information on scheduling for seeing a professional for work or assistance. As an example, waiting rooms can pose hazards from infection by contact with other infected individuals for people waiting to see a doctor. The present invention solves this problem, and others, by providing a system for real time updates to patients on the progress of a professional, making it safer for both the patients/customers and the attending professional by limiting waiting room times as well as allowing patients/customers to better allocate arrival times.

The present invention is distinguished from the following prior art:

U.S. Pat. No. 5,823,948 discloses a pattern of automatic generation of medical record as consequence of data entry. The present invention is distinguished from '948 and discloses a system that doesn't have any automatic report generation mechanism for medical records.

U.S. Pat. No. 6,988,075 discloses patient/customer records available by remote access by both patients and medical providers. The present invention is distinguished from '075 and discloses a system that doesn't provide any remote access to patients for medical records.

U.S. Pat. No. 8,244,566 discloses an automated online appointment scheduling service. The present invention is distinguished from '566 and discloses a system that supports real time manual adjustments in a patient's schedule based on data input.

U.S. Pat. No. 8,560,371 discloses suggesting activities to be performed during time slots of a user schedule. The present invention is distinguished from '371 and discloses a system which only supports manual changes in the patient's scheduling, so there is no suggestion of activities to be performed during time slots.

U.S. Pat. No. 9,171,252 discloses a system using a message broker. The present invention is distinguished from '252 and discloses a system for working on up-to-date real time schedule modifications specifically for the patient/customer.

U.S. Pat. No. 9,262,732 discloses a system for a support enterprise data processing environment for tracking action items. The present invention is distinguished from '732 as it does not involve the tracking of items, but rather enables real-time schedule modifications.

U.S. Pat. No. 9,552,560 discloses a system for user event notification to notify the user of an event before a starting time. The present invention is distinguished from '560 as it triggers a notification which enables scheduling and rescheduling.

U.S. Pat. No. 9,886,727 discloses a process that is completely automated with no user intervention once the process is started. The present invention is distinguished from '727 as it requires user input during the execution of the process.

U.S. Pat. No. 10,088,973 discloses presenting a set of intention objects arranged by course time windows. The present invention is distinguished from '973 as it does not support a graphical representation of a schedule process.

US 2008/0133290 discloses a use to customize the report that is generated from the information that is computed and compiled by the physician quality information system, not a scheduling module. The present invention does not use automatic report generation mechanisms, but does real time scheduling adjustments.

US 2008/0299960 discloses a message transmission manager that is configured to allow a user to select one or more transmission criteria for a first message that is to be transmitted using at least one messaging application, but not scheduling information. The present invention does not use a message transmission based on certain threshold value.

US 2010/0269049 discloses a method for managing events in a multiple schedule environment with user interaction and the user selection identifying an event that is to be scheduled. The present invention does not support a multiple scheduling environment; also there is no suggestion against time slot.

US 2011/0246226 discloses a pattern comprised of a section where a user adds and removes clinical and administrative flags that are associated with patients and that are displayed as reminders when the patients schedule visits, said clinical flags alerting the user to a patient's medical issue that requires special attention. The present invention does not have any clinical information and/or administrative flags that are associated with patients and that are displayed.

US 2013/0325493 discloses a pattern generated 3-D model of the human body (avatar) created by aggregating personal health and medical data of a user. The present invention does not support generating a 3D model of the human body from user medical data.

US 2014/0337751 discloses techniques for automatically creating calendar items and related activity. The present invention does provide any automatic calendar event creation, rather it provides real time calendaring updates.

US 2015/0365480 discloses systems for communicating with electronic devices that are provided, as well as a method for interacting with electronic devices which may include a method for interacting with electronic devices on a network. The present invention does not focus on interactions between electronic devices.

US 2016/0335686 discloses a real-time management method, system, and mobile device which includes checking in a person with an associated mobile device, as well as monitoring and updating an experience associated with the person at the site during a duration the person is at the site. The present system is not focused on user experience management.

US 2016/0342955 discloses patterns that are directed to multi-entity calendar and time management displays and methods of use. The present invention does not focus on calendaring event=setup, but rather real time calendar modifications.

WO 2014/160777 discloses a system that incorporates a mobile user interface device allowing user inputs or detected events from a variety of medical instrumentation devices to be controlled by a healthcare web server computer over a computer communication network. The present invention does not focus on monitoring patients.

### SUMMARY

In several embodiments, the present invention is systems and methods for real time updating of appointment schedules for appointment making professionals, including medical and dental applications. In several embodiments, the present invention is a system in which an intake clerk can manage a patient/customer's knowledge of a professional's scheduling, and at the same time send real time updates for appointment delays to scheduled patients.

In several embodiments, the present invention is a real-time appointment management system comprising: a new appointment module for assigning a time to a professional; a professional creation module for assigning professional intervals and SMS intervals; a data transmission module for sending information in said new appointment module to an end user; a database for storing updates on information supplied to, and from, the new appointment module and professional creation module in real-time; a transmitter for sending data stored in said database to an end user data reception device. In several embodiments, the real-time appointment management system has a professional list added to said professional creation module. In several embodiments, the real-time appointment management system has said database populated with information that includes at least professional completion time and professional intervals. In several embodiments, the real-time appointment management system has said information sent from said database to said end user data reception device which includes information in regard to a professional's delays as well as progress.

In several embodiments, the present invention is a method for real-time appointment management comprising the steps of: initiating a new appointment module for assigning a time to a professional; initiating a professional creation module for assigning professional intervals and SMS intervals; obtaining a data transmission module for sending information in said new appointment module to an end user; obtaining a database for storing updates on information supplied to, and from, said new appointment module and professional creation module in real-time; transmitting data stored in said database to an end user data reception device. In several embodiments, the real-time appointment management system has the additional step of adding a professional list to said professional creation module. In several embodiments, the real-time appointment management system has the additional step of populating said database with information that includes at least professional completion time and professional intervals. In several embodiments, the real-time appointment management system has the additional step of said information sent from said database to said end user data reception device which includes information in regard to a professional's delays.

In several embodiments, the present invention is a real-time appointment management system comprising: a client device with an internet connection to either receive or supply data; and a data repository to send and receive information from said internet connection; said data repository further comprises: an interface module; said interface module further comprises a web server and an API server; an application server; said application server further comprising appointments, professionals and a SMS Module; a database server; and a database.

### BRIEF DESCRIPTION OF THE DRAWINGS

For a more complete understanding of the present disclosure and the advantages thereof, reference is now made to the following descriptions to be taken in conjunction with the accompanying drawings describing specific embodiments of the disclosure, wherein:

FIG. 1 is a flow chart of one embodiment of the present invention for an appointment.

FIG. 2 is a flow chart of one embodiment of the present invention for a professional.

FIG. 3 is a flow chart of one embodiment of the present invention for an inputting user.

FIG. 4 is a flow chart of another embodiment of the present invention for a professional.

FIG. 5 is a flow chart of one system embodiment of the present invention.

### DETAILED DESCRIPTION

One or more illustrative embodiments incorporating the invention disclosed herein are presented below. Applicant has created revolutionary and novel systems and methods for real time updating of appointment schedules in preferably medical and dental applications.

In the following description, certain details are set forth such as specific quantities, sizes, etc. so as to provide a thorough understanding of the present embodiments disclosed herein. However, it will be evident to those of ordinary skill in the art that the present disclosure may be practiced without such specific details. In many cases, details concerning such considerations and the like have been omitted inasmuch as such details are not necessary to obtain a complete understanding of the present disclosure and are within the skills of persons of ordinary skill in the relevant art.

Referring to the drawings in general, it will be understood that the illustrations are for the purpose of describing particular embodiments of the disclosure and are not intended to be limiting thereto. Drawings are not necessarily to scale, and arrangements of specific units in the drawings can vary.

While most of the terms used herein will be recognizable to those of ordinary skill in the art, it should be understood, however, that when not explicitly defined, terms should be interpreted as adopting a meaning presently accepted by those of ordinary skill in the art. In cases where the construction of a term would render it meaningless, or essentially meaningless, the definition should be taken from Webster's Dictionary, New Edition, 2020. Definitions and/or interpretations should not be incorporated from other patent applications, patents, or publications, related or not, unless specifically stated in this specification or if the incorporation is necessary for maintaining validity. As defined herein, the following terms may have the following meanings: “Professional(s)”, as defined herein, can include: “medical, dental; or any licensed or unlicensed professional for services.” “Appointment(s)”, as defined herein, can include: “any scheduled appointment with a professional.” “Interval(s)”, as defined herein, can include: “any time duration for which a professional schedules a professional service to be performed.” “Calculate the delay”, as defined herein, can include: “the time difference between a scheduled appointment and the real time delay in which a professional can perform the scheduled appointment.” “Database(s)”, as defined herein, can include: “a physical database or cloud based.” “Client device”, as defined herein, can include: “a laptop, a smart device, smart tablet, or desk top computer.” “Web Client”, as defined herein, can include: “any browser.” “Interface Module”, as defined herein, can include: “a Web Server and an API server.” “API Server”, as defined herein, can include: “an application programing interface.” “SMS Module”, as defined herein, can include: “a text messaging system or other electronic mail system.” “User”, as defined herein, can include: “an end user, a patient/customer, or an intake clerk.” “Patient”, as defined herein, can include: “a doctor, or dental patient, as well as any customer of any professional”.

Certain terms are used in the following description and claims to refer to particular system components. As one skilled in the art will appreciate, different persons may refer to a component by different names. This document does not intend to distinguish between components that differ in name but not function. The drawing figures are not necessarily to scale. Certain features of the invention may be shown exaggerated in scale or in somewhat schematic form, and some details of conventional elements may not be shown, all in the interest of clarity and conciseness.

Although several preferred embodiments of the present invention have been described in detail herein, the invention is not limited hereto. It will be appreciated by those having ordinary skill in the art that various modifications can be made without materially departing from the novel and advantageous teachings of the invention. Accordingly, the embodiments disclosed herein are by way of example. It is to be understood that the scope of the invention is not to be limited thereby.

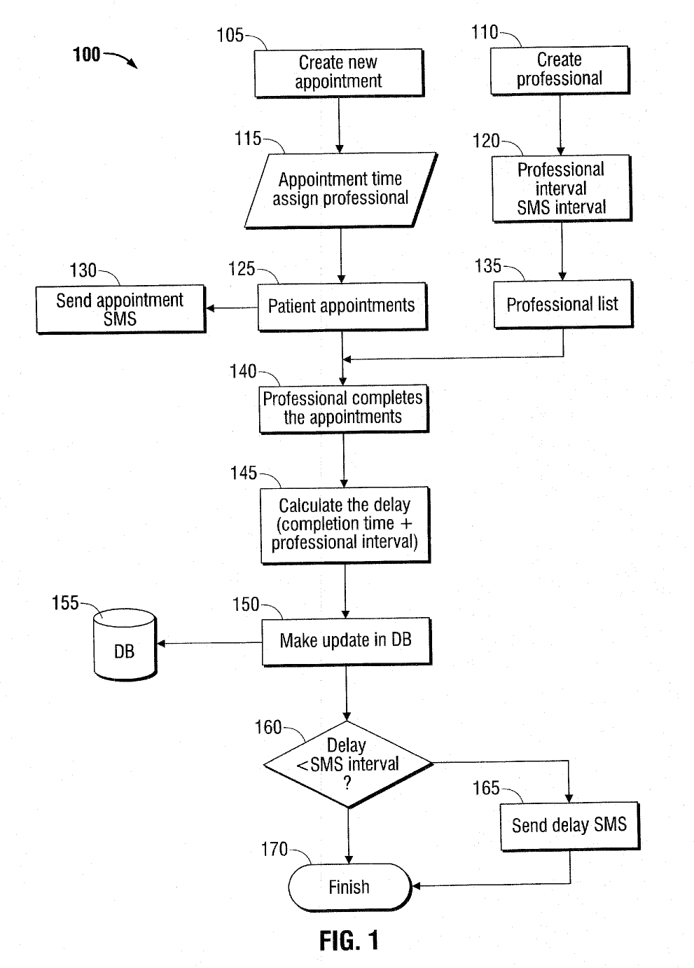

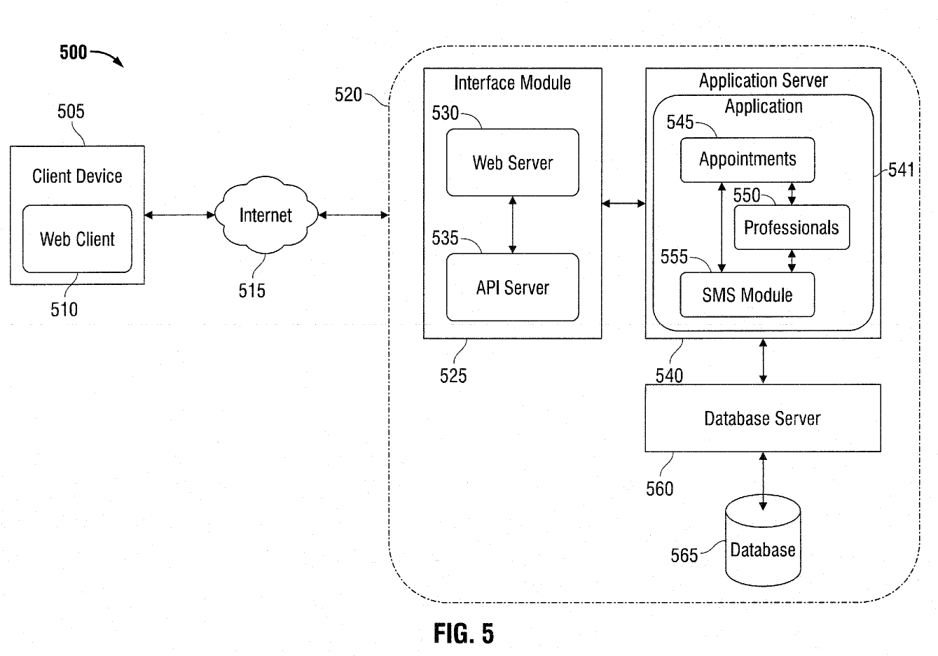

As shown, in several embodiments, FIG. 1 is a flow chart of one embodiment of the present invention for an appointment. As shown is one embodiment of the present invention for the completion of the creation of an appointment 100. As shown, in several embodiments, a user creates a new appointment 105. The new appointment 105 can be created through a user personal electronic device or on a computer which can relay information from the internet to an interface module and an application server (See FIG. 5). In several embodiments, the new appointment can be manually input via telephone and/or by a professional's staff. Next an appointment time is assigned to a professional 115. In several embodiments, this is accomplished by a review of date in the application server (FIG. 5) in order to ascertain availability for a professional and to have the information processed.

In several embodiments of the invention, a patient/customer appointment is created 125. In several embodiments of the present invention, this information is then relayed via the internet back to a user device or via SMS (FIG. 5). After the patient/customer appointment has been uploaded to the database server (FIG. 5) the appointment is sent via SMS 130 to an end user, and it can also be sent to an intake clerk. In several embodiments, an appointment can also be sent via e-mail or other electronic methods.

In several embodiments a “professional” is created 110 wherein professional information is input into the system, including, but not limited to, name, address, phone, contact information and other identifying information. In several embodiments, the “professional” is created by an intake clerk prior to any interaction with a patient/customer. Next a professional interval and SMS interval are created 120. Next a professional list is formed 135 which provides professional information and availabilities to see patients/customers. In several embodiments, after the professional list 135 is formed and the patient/customer appointments 125 are formed, the information is combined to the professional list to complete the appointment(s) 140. In many embodiments, the professional, professional's office, or intake clerk will input real time as to the length of professional visits with patients/customers, so accurate estimations of delays are provided to patients through SMS messages, or other electronic delivery methods.

In several embodiments, after the professional completes the appointments 140, next the system calculated the delay for a visitation (completion time+professional interval) 145. In several embodiments, this functions by the professional, or the professional's staff, inputting data that is transmitted to the system to indicate the competition of a visit, or event, wherein the system will update the database with real time completion records. So, in several embodiments, next, information is updated 150 in the information database 155. In several embodiments, information is assessed to determine if the delay is less than the SMS interval 160. In some embodiments, if there is a delay, then a delay message is sent to the SMS 165. If there is no delay, then the system is finished 170.

In several embodiments, by having real time input by a professional, or professional's office, information can be relayed to an end user through the internet, e-mail, and/or SMS and to a user device providing real time scheduling. This real time information enables the patient/customer to adjust his/her arrival time and reduce time in the waiting area, which will subject a patient/customer to less potential exposure to infected individuals.

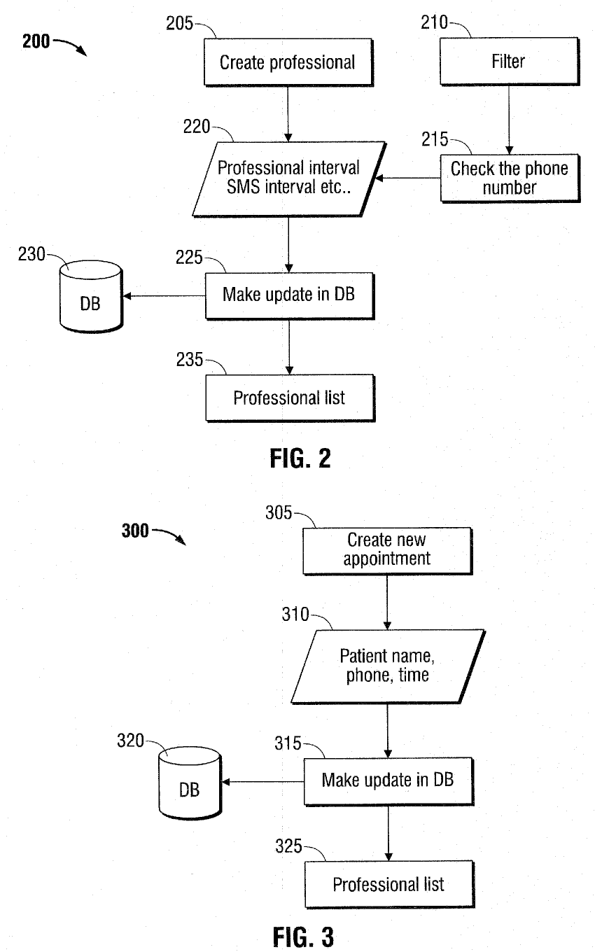

As shown, in several embodiments, FIG. 2 is a flow chart of one embodiment of the present invention for a professional creation for entry of data in the invention. As shown is one embodiment of a professional creation flow chart 200. In several embodiments, a “professional” is created 205. This creation can be done by an intake clerk, and will usually involve input of data such as name, phone number, practice, insurance, contact, or other standard inputs.

A filter 210 can also be applied next, and active phone number can be provided 215. This information can be obtained from an end user or another information source. In some embodiments, the filter 210 is designed to ensure that proper data for the professional is entered into the system. The system can receive and supply information on the professional's appointment scheduling. The information of the professional creation 205 and the phone number 215 is then combined to create a professional interval and SMS interval 220. In some embodiments, information from the professional interval/SMS interval 220 is sent as updates 225 to database 230. In some embodiments, after the update, the professional list 235 is then generated. In embodiments of the present invention, the updates are inputted and transmitted in real time, therein allowing for increased accuracy as data is sent to an end user device to view appointment updates (FIG. 5).

As shown, in several embodiments, FIG. 3 is a flow chart of one embodiment of the present invention for an appointment creation, for a patient/customer intake. As shown is one embodiment of an appointment creation flow chart 300. In several embodiments, a user can create a new appointment 305. In several embodiments, the user will input this information into a client device, or as a web client (FIG. 5). In several embodiments, the user can be an intake clerk. Next, a patient/customer name, phone number and time for appointment can be entered 310. In some embodiments, information from the patient/customer name, phone number and time 310 are sent as updates 315 to database 320. In some embodiments, after the update, the professional list 325 is then generated. The professional list can be created by a matching of patient/customer information with professionals currently subscribed to the inventive real time database. (FIG. 5).

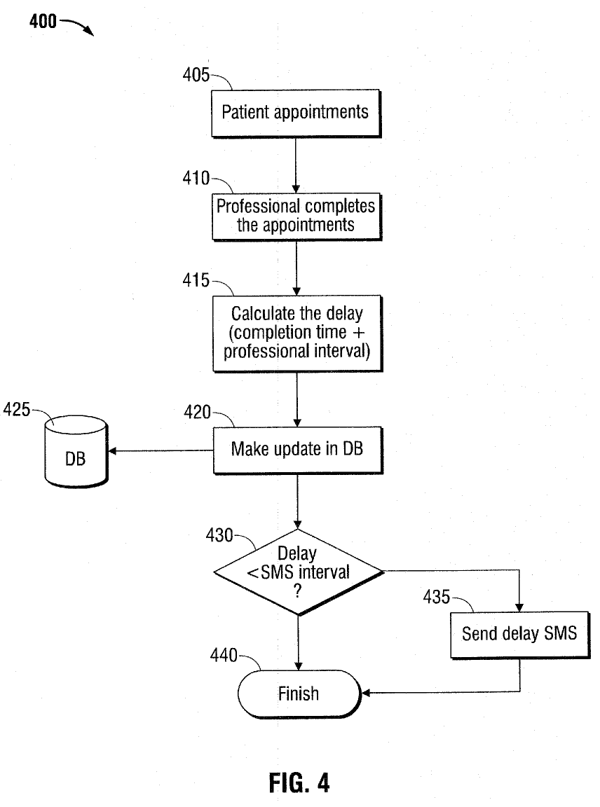

As shown, in several embodiments, FIG. 4 is a flow chart of another embodiment of the present invention for a professional after completion of initial entry into the database. As shown, is one embodiment of an appointment creation flow chart 400. As shown, in several embodiments, a patient/customer has an appointment which is commenced 405. A professional can then complete the appointment 410, and the completion time is entered into the database as a real time completion. In several embodiments, next the system calculates the delay (completion time+professional interval) 415 such that the lag time of the scheduled appointment is compared to the actual appointment time. In some embodiments, this information is updated 420 into a database 425. The updated information will then be transmitted as a delay message via the SMS interval 430. In several embodiments, if there is a delay in appointment scheduling, the delay is sent via SMS 435, and then the process is finished 440. In some embodiments, the process is finished 440 if there is not a delay.

In several embodiments of the present invention, by way of example, if a professional has a patient/customer scheduled for 11:00, that information will be stored in the database. If the professional is delayed by seeing patients before 11:00, the 11:00 patient/customer will receive periodic updates to their personal device as delays occur. So if the professional is delayed 10 minutes getting to a patient/customer at 9:00, all patients, including the one at 11:00, will receive a real time statement of the delay and the estimate for their own appointment change; in this case it would change to 11:10. If serious delays or cancellations occur, this information can also be provided to a patient/customer. By way of example, a 10-minute delay coupled with a cancellation, could equate to an appointment in the future running as originally scheduled. The real time updates on appointment scheduling, especially in the COVID pandemic of 2020, will provide massive safety improvements for physicians and patients alike by limiting waiting room crowding and times, thus limiting unnecessary exposure to other potentially sick individuals.

FIG. 5 is a flow chart of one system embodiment of the present invention. As shown data input/extraction 500 can occur in some embodiments of the present invention when a client device 505 or web client 510 interfaces through the internet (or other electronic method such as a phone) 515 to either receive or supply data. In some instances the interface can involve a user setting up, or checking, an appointment. In some instances, the interface can involve an input clerk or a professional. In some embodiments, there is a data repository 520 which can send and receive information from internet 515. In some instances, the user can be an input clerk, and in others it can be an end user.

In several embodiments of the present invention, data repository 520 can contain an interface module 525. In several embodiments of the present invention, the interface module 525 can contain web server 530 and API server 535. In several embodiments of the present invention, web server 530 and API server 535 can exchange data and information. In several embodiments of the present invention, there is an application server 540. In several embodiments, the application server has an application 541 that contains data on appointments 545, professionals 550 and an SMS Module 555. In several embodiments of the present invention, appointments 545, professionals 550 and SMS Module 555 all exchange and receive data in real time inputs. In several embodiments, there is a database server 560 which can exchange information with both a database 565 and the application server 540.

In several embodiments, information on real time scheduling changes is transmitted to an end user (client device 505) through the data repository 520, which is based upon real time inputs of professional appointment completions in view of actual scheduling times, as stored in the database 565. This update, in real time, can increase a professional's efficiency as well as cut down patient/customer waiting time as a result of accurate time keeping. If patients/customers are supplied information in real time, then they can allocate their schedules accordingly, and spend less time in waiting rooms with other potentially sick or infirm people.

In several embodiments, the present invention is a real-time appointment management system comprising: a new appointment module for assigning a time to a professional; a professional creation module for assigning professional intervals and SMS intervals; a data transmission module for sending information in said new appointment module to an end user; a database for storing updates on information supplied to, and from, the new appointment module and professional creation module in real-time; a transmitter for sending data stored in said database to an end user data reception device. In several embodiments, the real-time appointment management system has a professional list that is added to said professional creation module. In several embodiments, the real-time appointment management system has said database which is populated with information that includes at least professional completion time and professional intervals. In several embodiments, the real-time appointment management system has said information sent from said database to said end user data reception device which includes information in regard to a professional's delays.

In several embodiments, the present invention is a method for real-time appointment management comprising the steps of: initiating a new appointment module for assigning a time to a professional; initiating a professional creation module for assigning professional intervals and SMS intervals; obtaining a data transmission module for sending information in said new appointment module to an end user; obtaining a database for storing updates on information supplied to, and from, said new appointment module and professional creation module in real-time; transmitting data stored in said database to an end user data reception device. In several embodiments, the real-time appointment management system has the additional step of adding a professional list to said professional creation module. In several embodiments, the real-time appointment management system has the additional step of populating said database with information that includes at least professional completion time and professional intervals. In several embodiments, the real-time appointment management system has the additional step of said information sent from said database to said end user data reception device which includes information in regard to a professional's delays.

While preferred embodiments have been shown and described, modifications thereof can be made by one skilled in the art without departing from the scope or teaching herein. The embodiments described herein are exemplary only and are not limiting. Many variations and modifications of the system and apparatus are possible and will become apparent to those skilled in the art once the above disclosure is fully appreciated. For example, the relative dimensions of various parts, the materials from which the various parts are made, and other parameters can be varied.

I claim:

1. A real-time appointment management system comprising:
a new appointment module for assigning a time to a professional;
a professional creation module for assigning professional intervals and SMS intervals;
a data transmission module for sending information in said new appointment module to an end user;
a database for storing updates on information supplied to, and from, the new appointment module and professional creation module in real-time;
a transmitter for sending data stored in said database to an end user data reception device.

2. The real-time appointment management system of claim 1 further comprising:
a professional list is added to said professional creation module.

3. The real-time appointment management system of claim 1 further comprising:
said database is populated with information that includes at least professional completion time and professional intervals.

4. The real-time appointment management system of claim 1 further comprising:
said information sent from said database to said end user data reception device includes information in regard to a professional's delays.

5. A method for real-time appointment management comprising the steps of:
initiating a new appointment module for assigning a time to a professional;
initiating a professional creation module for assigning professional intervals and SMS intervals;
obtaining a data transmission module for sending information in said new appointment module to an end user;
obtaining a database for storing updates on information supplied to, and from, said new appointment module and professional creation module in real-time;
transmitting data stored in said database to an end user data reception device.

6. The method of claim 5 further comprising:
adding a professional list to said professional creation module.

7. The method of claim 5 further comprising:
populating said database with information that includes at least professional completion time and professional intervals.

8. The method of claim 5 further comprising:
said information sent from said database to said end user data reception device includes information in regard to a professional's delays.

9. A real-time appointment management system comprising:
a client device with an internet connection to either receive or supply data; and
a data repository to send and receive information from said internet connection;
said data repository further comprises:
an interface module;
said interface module further comprises a web server and an API server;
an application server;
said application server further comprising appointments,
professionals and a SMS Module;
a database server; and
a database.
